(19) United States
(12) Patent Application Publication
(10) Pub. No.: US 2010/0106048 A1
(43) Pub. Date: Apr. 29, 2010

(54) TEMPERATURE MEASUREMENT AND METHOD FOR PERFORMING THE SAME

(76) Inventor: Robert Leonard Krullaards, Leidschendam (NL)

Correspondence Address: YOUNG & THOMPSON, 209 Madison Street, Suite 500, Alexandria, VA, 22314, US

(21) Appl. No.: 12/522,607

(22) PCT Filed: Jan. 9, 2008
(86) PCT No.: PCT/NL2008/050014

§ 371 Date: Jul. 14, 2009

(30) Foreign Application Priority Data
Jan. 9, 2007 (NL) 2000424

(51) Int. Cl. A61B 5/01; G01K 5/22

(52) U.S. Cl. 600/549

(57) ABSTRACT

Implement and method for determining the temperature of an extremity of a living being, as a result of which the load-bearing capacity of the relevant body part can be determined. This is based on the flow rate of the blood in the relevant extremity. It is proposed to provide everyday objects with a temperature sensor which may be a contact sensor or radiation sensor and, in a range between 27.5 and 30.5° C., emits a signal which differs from a signal when the temperature of the relevant extremity is outside this range. Surprisingly, it has been found that if the temperature is within the range, optimum conditions for load-bearing capacity of the relevant extremity are given. Parts of this type which may be gripped by the extremity may include a wide range of parts.

The present invention relates to a part held by an extremity comprising a temperature sensor and temperature-indicating means controlled by said sensor. Such a part is generally known in the prior art. Various constructions have been proposed to measure temperature of an extremity and thus to determine the condition thereof According to some opinions, the blood circulation of an extremity will be poor when the skin's temperature is too low and this will effect the performance of the extremity. Furthermore, it is assumed that such poor blood circulation is in part caused by tenseness. Thus, providing an indication of the temperature may have a self-teaching effect for the user or give an indication for the treating specialist.

However, it has been found that it is not always possible to provide an accurate and reliable reading. That is to say, sometimes the user receives a signal indicating that he should adjust his posture in a certain way while this is not necessary, whereas in other cases he does not receive a signal when he should have. It is an object of the present invention to provide a product which can be produced in a simple manner, by means of which the posture of the individual in question can be corrected in a simple manner.

This object is achieved by means of a part which is held by an extremity, having a hand palm temperature sensor as well as temperature-indicating means controlled by said sensor, said temperature-indicating means being designed to emit a first signal in a temperature range below a single fixed value and to emit a second signal in a temperature range above said predetermined fixed value, wherein the measuring accuracy with respect to said predetermined value is ±0.5° C. and preferably less than ±0.1° C. and said fixed value is in the temperature range of 27.5-30.5° C.

Surprisingly, it has been found that if the very accurately selected predetermined temperature described above is adhered to as reference, very accurate predictions can be made with regard to the behavior of the individual in question. The value of the respective predetermined temperature depends on the body part which is measured. By means of research, it is possible to determine the optimum temperature for a specific body part.

It should be noted that a temperature sensor is known from U.S. Pat. No. 4,468,137. However, it is not clear from this publication what the purpose of the temperature measurement is and, in addition, there are a large number of threshold values if the transitions from one cell to another cell are seen as threshold values.

In U.S. 2003/0086474, a temperature indicator is present which can applied to a spray can in order to check if the latter is ready for use. In this case, the temperature of the contents of the spray can is measured and this patent application does not relate to measuring the temperature of the palm of the hand and to inferring conclusions with regard to the physical condition of the individual in question. GB 2165646 describes a further temperature indicator.

The term hand palm sensor is understood to mean any sensor by means of which the temperature of the palm of the hand can be determined.

The temperature can be measured in various ways. Thus, it is possible to design the sensor as a contact heat sensor. However, it is also possible to design the sensor as radiation heat sensor. Instead of these electrical sensors, it is also possible to use chemical or physical sensors. Examples are colors which change at certain temperatures, for example on film material. In the case of electrical recording, it is possible to indicate whether the temperature is within or just outside the desired range in a similarly adjustable manner. This may be effected numerically or by colors, sounds, or any other method.

It will be understood that, for example if the tool with which the temperature is measured is a mouse, a PC offers endless possibilities of indicating the respective temperature and storing any values. Examples thereof are data being compared with previously stored data or advice being given by software, such as the advice that a better working posture/different working posture is desirable or that a period of rest is called for. In addition, it is also possible to take measures so that the PC becomes temporarily inoperative if the values are outside the optimum range.

The part described above can be incorporated into many objects of everyday use. Examples are parts which are held in the hand, such as handles of many objects, such as game means (rackets), joysticks and the like. It is also possible to provide appliances with simple temperature sensors which react in the abovementioned range. Consideration may be given to pens, writing implements, toothbrushes, tools, mice, telephones, joysticks, handles, steering members and other operating members, percussion instruments and other musical instruments and the like.

Another example are pieces of jewelry including watches. In the case of relatively large objects such as watches, it is possible for them to emit a signal if the temperature sinks below a certain value.

The invention can also be incorporated into garments, such as socks, gloves, surgical gloves, wristbands, insoles and the like. In the case of gloves, it is possible to carry out the measurement at the wrist.

It will be understood, that for the purposes of the present invention, the hollow of the foot corresponds to the palm of a hand.

The invention will be described in more detail below with reference to a number of examples, in which:

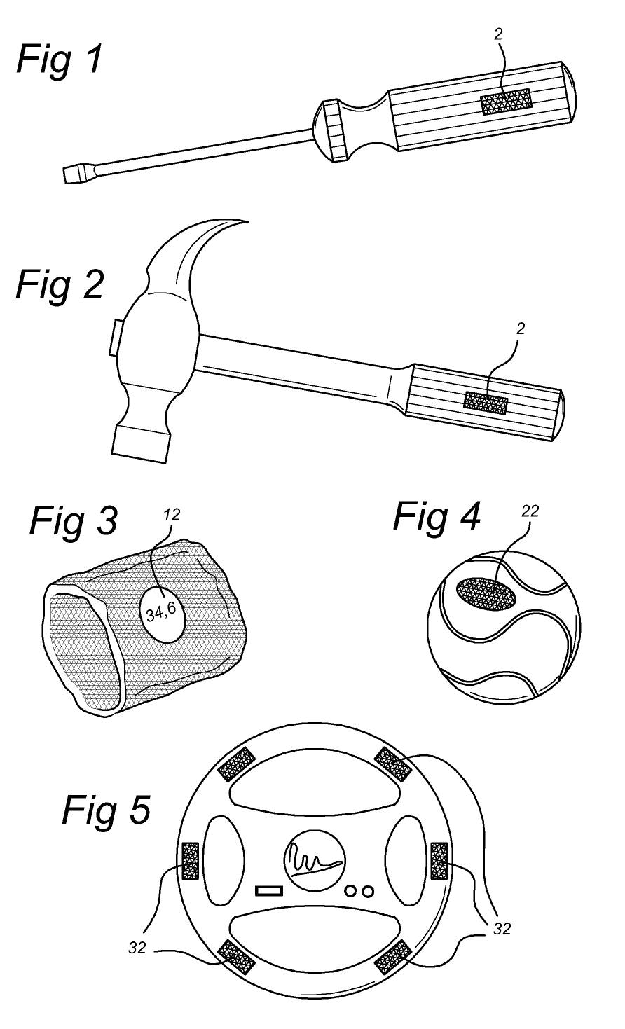

FIGS. 1-15 show various applications of the invention in a highly diagrammatic manner.

FIG. 1 shows a screwdriver and FIG. 2 a hammer. These are symbolic of the various tools in which the present invention can be used. Both of these are provided with a temperature sensor 2. This may be either a temperature sensor which is wirelessly connected to a computer system or an independently operating sensor. In the latter case, this sensor may comprise an electronic sensor, but it is also possible to use a sensor which operates on a chemical-physical basis, for example a sensor in which a change in color occurs at a certain increase in temperature.

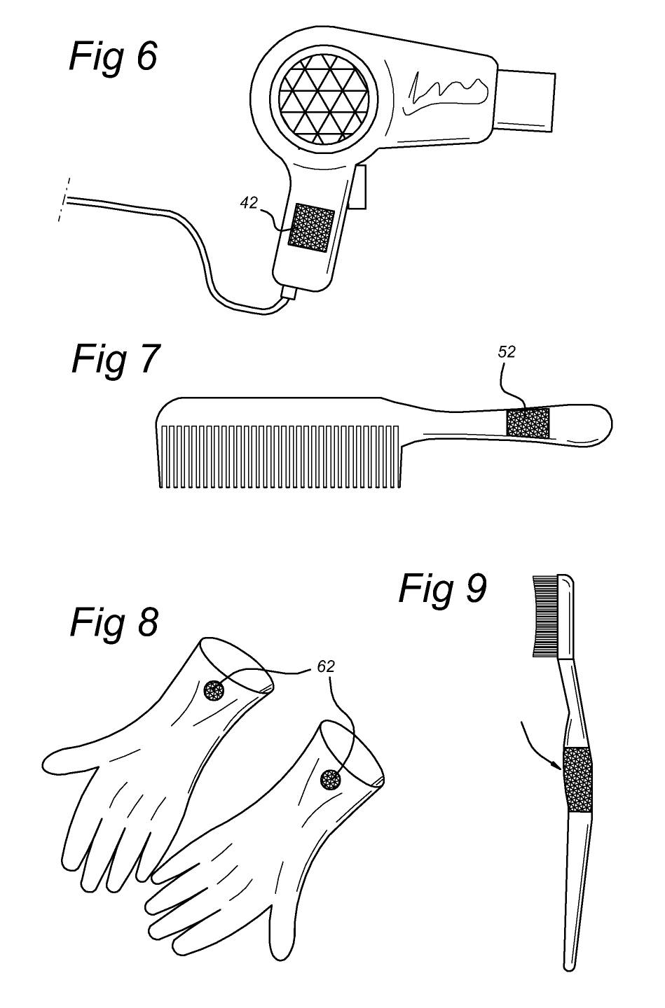

FIG. 3 shows a sweatband and the temperature sensor is indicated by reference numeral 12. FIG. 4 shows a stress ball and the temperature sensor is indicated by reference numeral 22. FIG. 5 shows a steering wheel or another operating device for a (motor) vehicle comprising the sensor 32. In this case as well, the sensor serves to indicate whether or not the conditions for the operator are optimum. FIG. 6 shows a hairdryer comprising a sensor 42 which is temperature-sensitive. FIG. 7 shows a temperature-sensitive sensor 52 in a comb. FIG. 8 shows a pair of gloves, such as surgical gloves, which are provided with a chemical-physical temperature sensor 62. It is also possible to use an electronic sensor which is connected to a computer system in the manner described below.

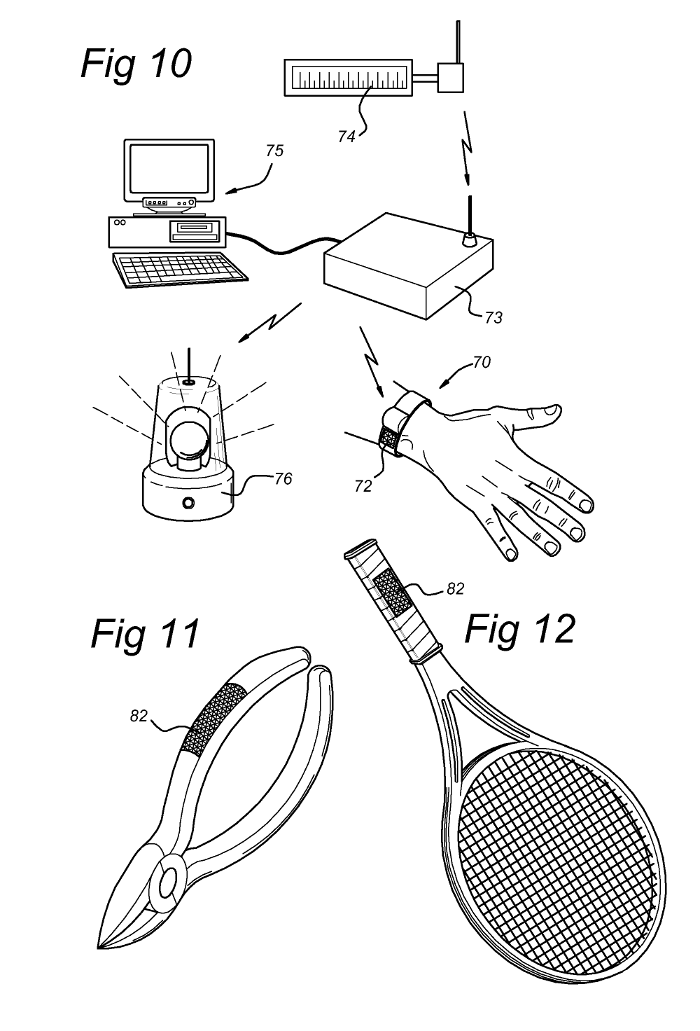

An example thereof is illustrated in FIG. 10, where a wristband 70 is provided comprising a temperature sensor 72 which is incorporated therein. The latter emits a signal which is characteristic of its temperature. This is transmitted to receiver 73. A signal coming from a thermometer 74 for the ambient temperature is likewise transmitted to the receiver 73. The receiver 73 is in communication with a computer 75 and, depending on the settings thereof, a signal will be emitted by the signal-emitting device 76 if the measured temperature is too low. FIG. 9 shows a toothbrush in which that part of the handle which is held by the user is provided with a temperature sensor.

The construction shown in FIG. 10 can also be used if the temperature is, for example, measured on a mouse or another device which is used in combination with a computer. An example thereof will be explained with reference to FIG. 15.

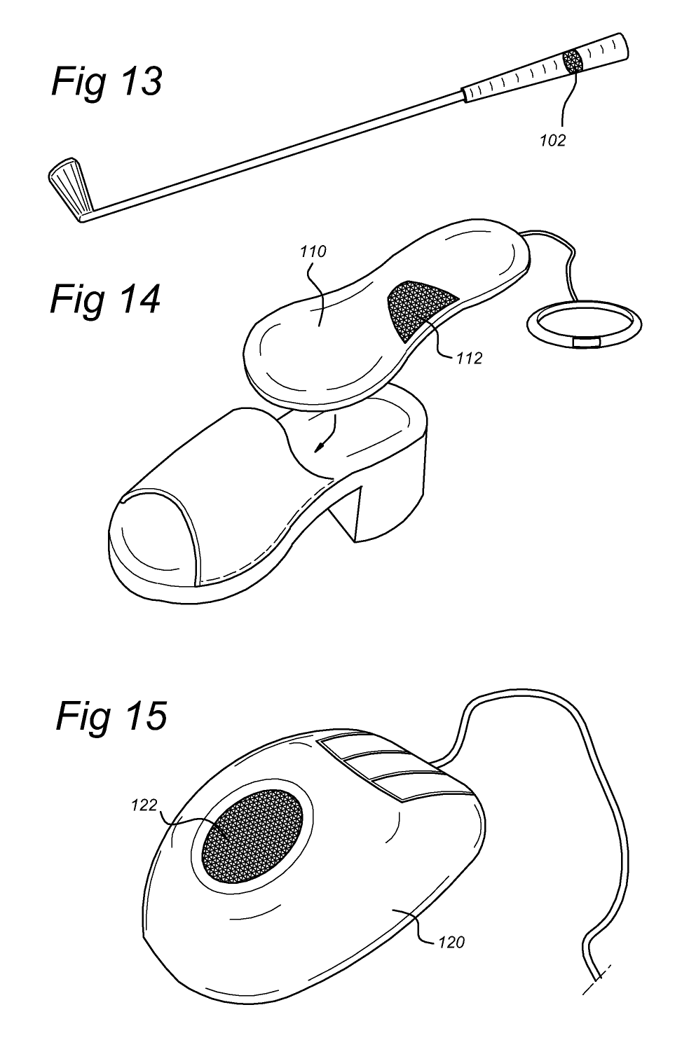

FIG. 11 shows a further tool, such as a pair of pliers, provided with a temperature sensor 82. FIG. 12 shows a tennis racket with a temperature sensor 92 while the golf club represented in FIG. 13 is provided with a temperature sensor 102. FIG. 14 shows the use of an insole in footwear and said insole is denoted by reference numeral 110. It is provided with a temperature sensor 112. The temperature measured at the foot can optionally be transmitted wirelessly.

FIG. 15 shows a mouse 120 provided with a temperature-sensitive part 122. This may be a part which simply reacts physically to temperature, such as films known from the prior art which change color at the relevant temperature. However, it is also possible to embody all this to operate electronically, resulting in a signal generated by a computer.

The above illustrates that there are numerous ways of putting the invention into practice. Those skilled in the art will immediately be able to think of further variants upon reading the above and these are deemed to be within the scope of the present invention.

1-11. (canceled)

12. An implement comprising a handle having a hand palm temperature sensor provided in the handle, and temperature-indicating means controlled by said sensor, said temperature-indicating means being designed to emit a first signal in a temperature range below a single fixed value and to emit a second signal in a temperature range above said single fixed value, wherein the measuring accuracy with respect to said fixed value is ±0.5° C. and said fixed value being in the temperature range from 27.5-30.5° C.

13. The implement as claimed in claim 12, wherein said sensor comprises a contact heat sensor.

14. The implement as claimed in claim 12, wherein said sensor is a radiation heat sensor.

15. The implement as claimed in claim 12, wherein said sensor is a sensor which changes color at said fixed value.

16. The implement as claimed in claim 12, comprising a garment.

17. The implement as claimed in claim 12, comprising an appliance such as a tool.

18. The implement as claimed in claim 12, comprising a game means.

19. The implement as claimed in claim 12, comprising footwear.

20. A method for determining the condition of the extremity of a living being, comprising a palm of a hand engaging a handle of an implement and determining the temperature of said palm of a hand at the spot of said held part, emitting a first signal if the detected temperature is below a single fixed value and emitting a second signal differing from the first if the temperature is above said single fixed value, wherein said fixed value is in the temperature range 27.5-30.5° C. and the measurement accuracy with regard to said fixed value is ±0.5.

21. The method as claimed in claim 20, wherein said temperature range is between 29° C. and 30° C.

22. The method as claimed in claim 20, wherein said fixed value is determined by measuring the temperature of a reference body part.
